(19) United States
(12) Patent Application Publication
(10) Pub. No.: US 2011/0281950 A1
(43) Pub. Date: Nov. 17, 2011

(54) COMPOSITIONS AND METHODS FOR REDUCING PROLIFERATION AND VIABILITY OF LYMPHOBLASTOID CELLS

(76) Inventor: Robert A. Baiocchi, Dublin, OH (US)

(21) Appl. No.: 13/105,576

(22) Filed: May 11, 2011

Related U.S. Application Data
(60) Provisional application No. 61/333,600, filed on May 11, 2010.

(51) Int. Cl. A61K 31/167; A61P 35/00

(52) U.S. Cl. 514/575

(57) ABSTRACT

Pharmaceutical compositions including an HDAC inhibitor and a pharmaceutically acceptable carrier wherein the HDAC inhibitor is present in an amount sufficient to achieve a plasma concentration from about 100 nM to about 2 uM and methods of treatment using the same. Pharmaceutical compositions including an HDAC inhibitor and a pharmaceutically acceptable carrier wherein the HDAC inhibitor is present in an amount from 0.1 mg to 100 mg. Pharmaceutical compositions including an HDAC inhibitor and a pharmaceutically acceptable carrier, wherein the concentration of the HDAC inhibitor is sufficient to decrease the relative viability of lymphoblastoid cells by at least about 50 percent and/or is sufficient to decrease the proliferation of lymphoblastoid cells by at least about 60 percent and/or is sufficient to decrease the relative viability of peripheral blood mononuclear cells by less than about 50 percent and methods of treatment using the same.

### CLAIM OF PRIORITY

This application claims priority under 35 U.S.C. §119(e) to U.S. Provisional Patent Application Ser. No. 61/333,600 filed on May 11, 2010, which is hereby incorporated by reference in its entirety.

### BACKGROUND

Immune deficiency is an ever increasing problem, made evident in recent years by the global HIV/AIDS epidemic, the fact that organ transplantation is now at an all time high, and a steadily approaching population explosion among geriatric patients. A myriad of complications accompany immune deficiency including an increased risk of developing cancer, especially virally-associated malignancies. Examples of these types of disorders include human papillomavirus (HPV)-associated cervical and skin cancer, hepatitis B and C-associated hepatocellular carcinoma, and human T-lymphotropic virus (HTLV)-associated adult T cell leukemia.

Epstein Barr virus (EBV) is a ubiquitous human herpes virus that infects B lymphocytes and epithelial cells. EBV can directly induce B cell transformation and is associated with a variety of neoplastic diseases that can each be identified by characteristic patterns of latent viral gene expression. High mortality rates associated with most EBV-associated malignancies highlight the need for effective treatment strategies. However, traditional cancer treatments like chemotherapy and radiation therapy are unsuitable for this purpose because they cause further immunologic compromise.

While antiproliferative therapy for cancers can be effective in treating various cancers, antiproliferative therapy is often associated with side effects including reduced immune function. Thus, patients undergoing chemotherapy are at increased risk for developing infections which complicate recovery from their condition. Thus, antiproliferative therapy that does not also compromise immune function is desirable.

### BRIEF DESCRIPTION OF THE DRAWINGS

FIG. 1 is a representative portrayal of epigenetic silencing.

FIG. 2 is schematic representation of a ⁵¹Cr release assay.

FIGS. 3A-3C are graphs showing experimental data depicting the percentage of viable B cells per days cultured, for various concentrations of AR-42, SAHA and VPA, respectively.

FIG. 4 is a chart showing experimental data of relative NK cytotoxicity of various concentrations of AR-42.

FIG. 5 is a chart showing experimental data of relative CTL cytotoxicity at various concentrations of AR-42 and various effector:target ratios.

FIGS. 6A-6D show experimental data for the number of visible spots counted at various concentrations of AR-42 for NK IFNγ, NK GzB, T cell IFNγ and T cell GzB, respectively.

FIG. 7 is a graph showing experimental data of mean fluorescent intensity (MFI) for various HDAC inhibitors.

FIGS. 8A and 8B are charts showing experimental data of number of visible spots counted (IFNγ and GzB) for various concentrations of AR-42 and washout samples

FIG. 9A is a chart showing experimental data of number of visible spots counted (IFNγ) of various concentrations of AR-42 and washout samples.

FIG. 9B is a chart showing experimental data of relative NK cytotoxicity of various concentrations of AR-42 and washout samples.

FIG. 9C is a chart showing experimental data of relative NK cytotoxicity of various concentrations of SAHA and VPA and washout samples.

FIG. 10 shows experimental data of an EBV-specific T-cell expansion.

FIG. 11 shows experimental data from an expansion comparing PBMCs not-stimulated with LCLs to PBMCs stimulated with LCLs.

FIG. 12 shows experimental data for EBV+ LCL incubated with AR-42 at 500 nM and 1000 nM for 24 and 48 hours.

FIG. 13 shows experimental data for relative cytotoxicity for various concentrations of AR-42.

FIG. 14A shows experimental data for relative viability of LCL lines at 4 days at various concentrations of AR-42.

FIG. 14B shows experimental data for relative cell proliferation at day 5 at various concentrations of AR-42.

FIG. 15 shows experimental data for relative viability of PBMCs at various concentrations of AR-42.

FIG. 16 shows experimental data for percentage of CD8+ T cells after 14 days of culture with irradiated LCLs at various concentrations of AR-42.

FIG. 17 shows experimental data for relative visible spots at various concentrations of AR-42.

FIG. 18 shows experimental data for relative cytotoxicity at various concentrations of AR-42.

FIG. 19 shows experimental data for relative cytotoxicity in MAC cells at AR-42 (1000 nM) and washout samples.

FIG. 20 shows experimental data for relative cytotoxicity in purified NK cells at various concentrations of AR-42.

FIG. 21 shows experimental data for mean fluorescent intensity versus AR-42 concentration describing surface marker expression in purified NK cells.

### DETAILED DESCRIPTION

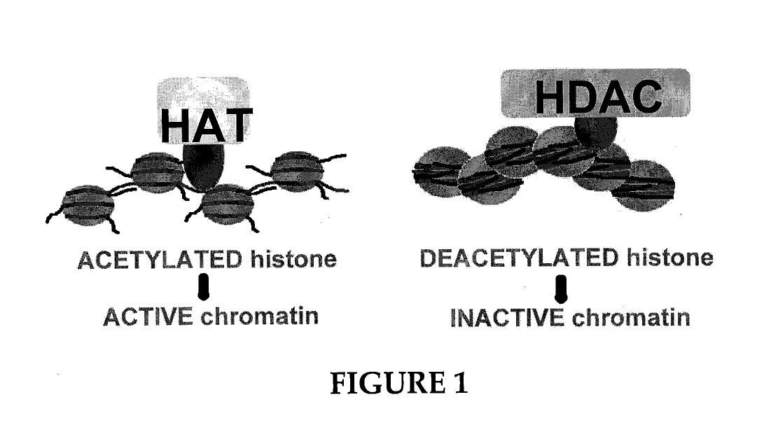

Transcriptional processes are tightly controlled and governed by multiple regulatory mechanisms in both the human and EBV genome. DNA that is tightly wrapped around localized aggregations of histones, called nucleosomes, is inaccessible to chromatin remodeling complexes and transcription factors. Thus, highly condensed chromatin facilitates “transcriptional silencing.” Nucleosomes are usually found in the DNA of eukaryotic cells but have also been discovered in the episomal genome of EBV. Post-translational (epigenetic) modification of EBV DNA can induce the expression of select genes in the EBV genome that are normally transcriptionally silent. Histone acetyltransferase (HAT) is an enzyme that epigenetically modifies genomic DNA. Histones have positively charged tails that bind electrostatically to negatively charged phosphate groups found on DNA nucleotides. HAT neutralizes the tails of histones and the resulting relaxation of the nucleosomal structure facilitates binding of transcription factors and ultimately, transcriptional activity. An opposing enzyme, histone deacetylase (HDAC) restores the positive charge onto the tails of histones and suppresses transcription. See, e.g., FIG. 1.

Epigenetic gene silencing has been associated with the onset and progression of a wide range of diseases, including cancer. HDAC enzymes influence the epigenome by covalently modifying histone tails promoting tighter nucleosomal structure and transcriptional silencing. Consequently, drugs that inhibit HDAC enzymes hold significant promise as specific and effective therapeutic agents.

Drugs that selectively inhibit key enzymes involved with transcriptional silencing of important regulatory and anti-cancer genes have been shown to be useful anti-tumor agents and may be used for treating EBV+ malignancies. In EBV transformed cells, HDAC activity is upregulated. Consequently, key tumor suppressor genes are silenced and transformed cells can evade host immune defenses by silencing the expression of surface markers used to target them. HDAC inhibitors not only induce apoptosis in EBV+ LCLs, but also make them more visible to host immune defenses by promoting expression of target genes that are “seen” by EBV-specific effector cells. HDAC inhibitors are non-toxic to non-transformed lymphocytes.

The HDAC inhibitors used in the various aspects describe herein include, but are not limited to, the molecules described in U.S. application Ser. No. 10/597,022, which is hereby incorporated by reference in its entirety, and are based on, for example, fatty acids coupled with Zn²⁺-chelating motifs through aromatic Ω-amino acid linkers. In various aspects, the HDAC inhibitors may have the formula:

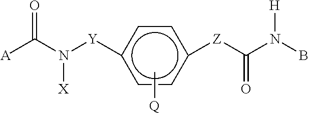

wherein X is chosen from H and CH₃; Y is (CH₂)n wherein n is 0-2; Z is chosen from (CH₂)ₘ wherein m is 0-3 and (CH)₂; A is a hydrocarbyl group; B is o-aminophenyl or hydroxyl group; and Q is a halogen, hydrogen, or methyl.

Specific examples of such inhibitors include, for example, N-(2-Amino-phenyl)-4-[(2-propyl-pentanoylamino)-methyl]-benzamide; N-Hydroxy-4-[(2-propyl-pentanoylamino)-methyl]-benzamide; N-(2-Amino-phenyl)-4-(2-propyl-pentanoylamino)-benzamide; N-Hydroxy-4-(2-propyl-pentanoylamino)-benzamide; 2-Propyl-pentanoic acid {-4-[2-amino-phenylcarbamoyl)-methyl]-phenyl}-amide; 2-Propyl-pentanoic acid (4-hydroxycarbamoyl-methyl-phenyl)-amide; 2-Propyl-pentanoic acid {4-[2-amino-phenylcarbamoyl)-ethyl]-phenyl}-amide; 2-Propyl-pentanoic acid [4-(2-hydroxycarbamoyl-ethyl)-phenyl]amide; 2-Propyl-pentanoic acid {4-2-(2-amino-phenylcarbamoyl)-vinyl]-phenyl}-amide; 2-Propyl-pentanoic acid [4-(2-hydroxycarbamoyl-vinyl)-phenyl]-amide; N-(2-Amino-phenyl)-4-(butyrylamino-methyl)-benzamide; N-(2-Amino-phenyl)-4-(phenylacetylamino-methyl)-benzamide; N-(2-Amino-phenyl)-4-[(4-phenyl-butyrylamino-methyl]-benzamide; 4-(Butyrylamino-methyl)-N-hydroxy-benzamide; N-hydroxy-4-(phenylacetylamino-methyl)-benzamide; N-hydroxy-4-[(4-phenyl-butyrylamino)-methyl]-benzamide; 4-Butyrylamino-N-hydroxy-benzamide; N-hydroxy-4-phenylacetylamino-benzamide; N-hydroxy-4-(4-phenylbutyrylamino)-benzamide; N-[4-(2-Hydroxycarbamoyl-ethyl)-phenyl]-butyramide; N-hydroxy-3-(4-phenylacetylamino-phenyl)-propionamide; N-[4-(2-Hydroxycarbamoyl-ethyl)-phenyl]-4-phenyl-butyramide; N-(2-Amino-phenyl)-4-[(2-phenyl-butyrylamino-methyl]-benzamide; N-(2-Amino-phenyl)-4-[(3-phenyl-butyrylamino-methyl]-benzamide; N-hydroxy-4-(2-phenylbutyrylamino)-benzamide; N-hydroxy-4-(3-phenylbutyrylamino)-benzamide; N-[4-(2-Hydroxycarbamoyl-ethyl)-phenyl]-2-phenyl-butyramide; N-[4-(2-Hydroxycarbamoyl-ethyl)-phenyl]-3-phenyl-butyramide; N-hydroxy-4-[(2-phenyl-butyrylamino)-methyl]-benzamide; N-hydroxy-4-[(3-phenyl-butyrylamino)-methyl]-benzamide; 4-Benzoylamino-N-hydroxy-benzamide; 4-(4-methyl)-Benzoylamino-N-hydroxy-benzamide; 4-(4-chloro)-Benzoylamino-N-hydroxy-benzamide; 4-(4-bromo)-Benzoylamino-N-hydroxy-benzamide; 4-(4-tert-butyl)-Benzoylamino-N-hydroxy-benzamide; 4-(4-phenyl)-Benzoylamino-N-hydroxy-benzamide; 4-(4-methoxyl)-Benzoylamino-N-hydroxy-benzamide; 4-(4-trifluoromethyl)-Benzoylamino-N-hydroxy-benzamide; 4-(4-nitro)-Benzoylamino-N-hydroxy-benzamide; Pyridine-2-carboxylic acid (4-hydroxycarbamoyl-phenyl)-amide; N-hydroxy-4-(2-methyl-2-phenyl-propionylamino)-benzamide; N-hydroxy-4-(3-methyl-2-phenyl-butyrylamino)-benzamide; N-hydroxy-4-(3-phenyl-propionylamino)-benzamide; 4-(2,2-Dimethyl-4-phenyl-butyrylamino)-N-hydroxy-benzamide; N-hydroxy-4-[methyl-(4-phenyl-butyryl)-amino]-benzamide; N-hydroxy-4-(2-phenyl-propionylamino)-benzamide; N-hydroxy-4-(2-methoxy-2-phenyl-acetylamino)-benzamide; 4-Diphenylacetylamino-N-hydroxy-benzamide; N-hydroxy-4-[2-(4-isobutyl-phenyl)-propionylamino]-benzamide; N-(2-Amino-phenyl)-4-phenylacetylamino-benzamide; N-(2-Amino-phenyl)-4-(5-phenyl-pentanoylamino)-benzamide; N-(2-Amino-phenyl)-4-(2-phenyl-butyrylamino)-benzamide; N-(2-Amino-phenyl)-4-(2,2-dimethyl-4-phenyl-butyrylamino)-benzamide; N-(2-Amino-phenyl)-4-(3-phenyl-propionylamino)-benzamide; N-(2-Amino-phenyl)-4-(4-phenyl-butyrylamino)-benzamide; N-(2-Amino-phenyl)-4-(3-phenyl-butyrylamino)-benzamide; N-(2-Amino-phenyl)-4-(3-methyl-2-phenyl-butyrylamino)-benzamide; N-(2-Amino-phenyl)-4-(2-methyl-2-phenyl-propionylamino)-benzamide; N-(2-Amino-phenyl)-4-[2-(4-isobutyl-phenyl)-propionylamino]-benzamide; and N-hydroxy-4-[2-(S)-phenylbutyrylamino]-benzamide; N-hydroxy-4-[2-(R)-phenyl butyrylamino]-benzamide; N-[4-(2-Hydroxycarbamoyl-ethyl)-phenyl]-2-(S)-phenyl-butyramide; N-[4-(2-Hydroxycarbamoyl-ethyl)-phenyl]-2-(R)-phenyl-butyramide; N-hydroxy-4-(3-(S)-phenylbutyrylamino)-benzamide; N-hydroxy-4-(3-(R)-phenylbutyrylamino)-benzamide; N-hydroxy-4-[3-(S)-phenylbutyrylamino]-benzamide; and N-hydroxy-4-[3-(R)-phenylbutyrylamino]-benzamide.

One HDAC inhibitor of particular interest is N-hydroxy-4-(3-methyl-2-phenyl-butyrylamino)-benzamide, and is also known as AR-42 (Arno Pharmaceuticals). The formula of AR-42 is as follows:

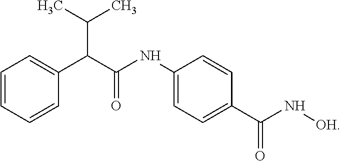

The HDAC inhibitor compounds used in the various aspects described herein may be racemates, or racemic mixtures. The term “racemic” as used herein means a mixture of the (R)- and (S)-enantiomers, or stereoisomers, of the compounds of the invention, in which neither enantiomer, or stereoisomer, is substantially purified from the other.

The term “enriched,” as used herein to describe (R)- or (S)-stereoisomers of the invention, refers to a composition having a greater amount of the (R)-stereoisomer than (S)-stereoisomer, or vice versa. For example, the composition may contain greater than 50%, 55%, or at least about 60% of the (S)-stereoisomer of AR-42 by weight, based on the total weight of AR-42. In one embodiment, the amount of enriched (S)-AR-42 may be higher, for example, at least about 65%, 70%, 75%, 80%, 81%, 82%, 83%, 84%, 85%, 86%, 87%, 88%, 90%, 91%, 92%, 93%, 94%, 95%, 96%, 97%, 98%, 99%, or any fraction thereof (i.e., 90.1%, 90.2%, etc.), of (S)-AR-42 by weight, based on the total weight of AR-42. In a particular embodiment, the amount of enriched (S)-AR-42 may be greater than 99%, 99.1%, 99.2%, 99.3%, 99.4%, 99.5%, 99.6%, 99.7%, 99.8%, 99.9% or may be 100%, by weight, based on the total weight of AR-42. These terms also define the amount of any pharmaceutically acceptable salts of (S)-AR-42. These are non-limiting examples, and the same enrichments may be achieved for other racemic compounds of the invention.

The effects of HDAC inhibitors, and more specifically of AR-42, on Epstein-Barr Virus (EBV)-transformed lymphoblastoid cell lines (LCLs) and on innate and adaptive immune functions are discussed in reference to Examples 1-14.

According to disclosed aspects, treatment with AR-42 provides antiproliferative activity with respect to tumor cells without permanent disruption of innate and adaptive immune functions. In other words, although treatment with AR-42 has an inhibitory affect on immune mechanisms, this inhibition is transient and reversible upon washout of AR-42. The term “washout,” in one aspect refers to removal of, or reduction in the concentration or bioavailability of a drug such as an HDAC inhibitor (e.g., AR-42).

Treatment with AR-42 provides several mechanisms by which antiproliferative activity with respect to tumor cells in increased. As can be seen from the experimental data in the various examples, low concentrations of AR-42 (e.g., 100 nM-1000 nM) provides antiproliferative activity with respect to tumor cells while having minimal affect on normal peripheral blood mononuclear cells (PBMCs). Washout of AR-42 allows for significant recovery of cytotoxic activity and IFNγ release from both NK and T cell effectors, showing that the immune inhibitory effect of AR-42 is transient and reversible. Thus, immune activity after washout may provide additional anti-tumor activity. Low concentrations of AR-42 also act to enhance the expression of proteins/targets that allow improved targeting of these antigens with antibodies after washout.

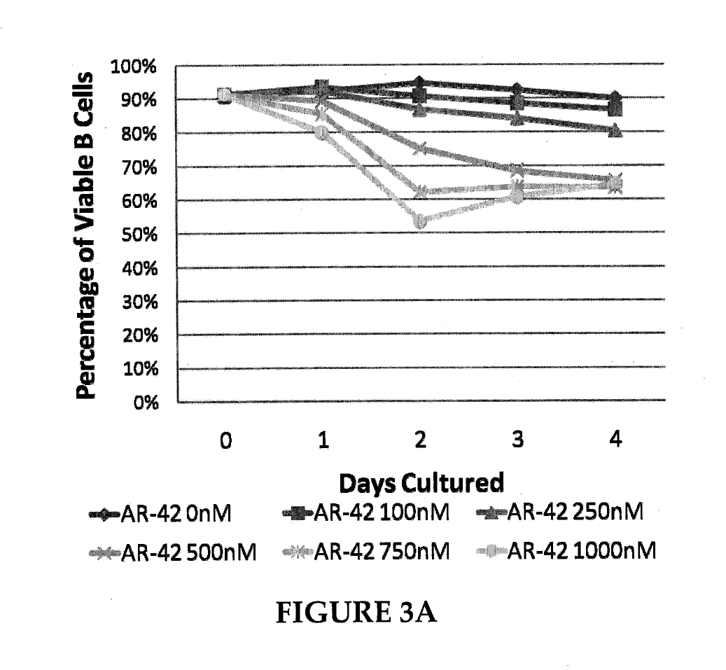

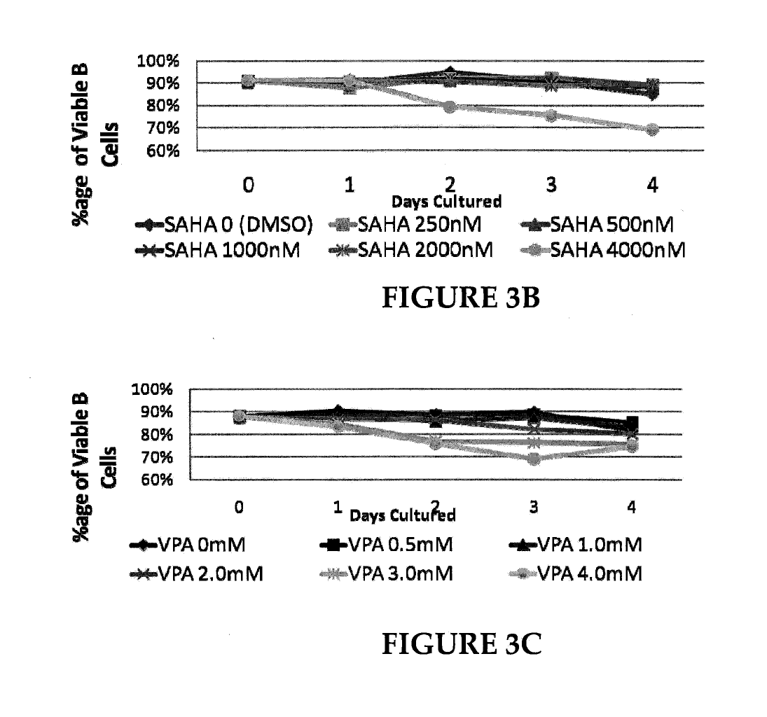

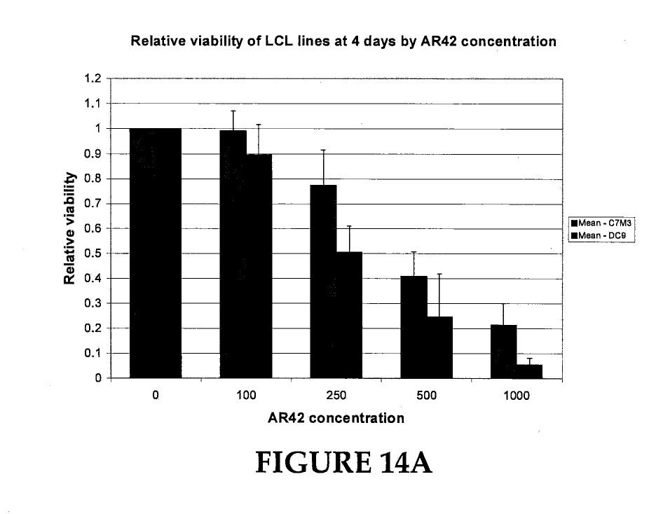

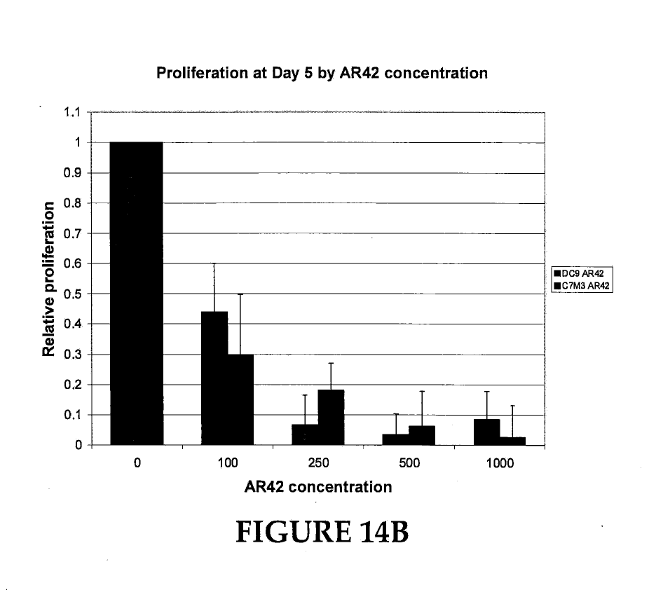

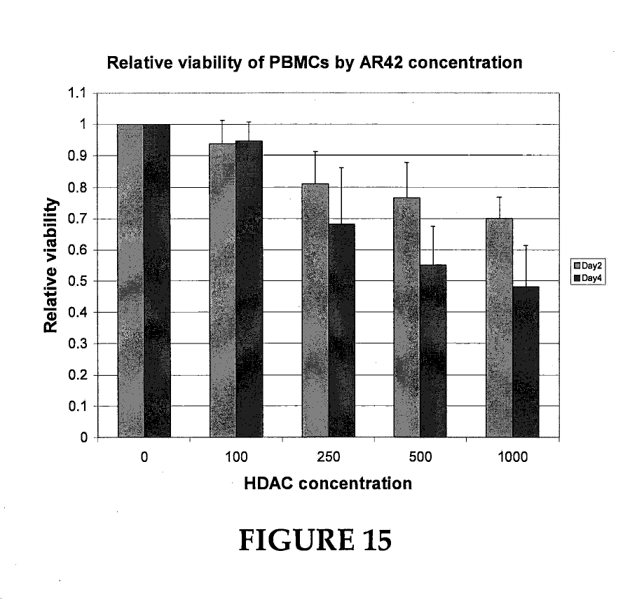

As seen in FIGS. 3A, 14A and 14B, AR-42 at concentrations of 100 nM, 250 nM, 500 nM, 750 nM and 1000 nM provide antiproliferative activity in EBV+ LCL cell lines in a dose dependent manner. At the same time, at these same concentrations of AR-42, the relative viability of PBMCs shown in FIG. 15 are higher as compared to the relative viability of the LCL lines (of FIG. 14A). Thus, it can be seen that AR-42 has direct antiproliferative effect on the LCLs without a significant relative effect on healthy cells.

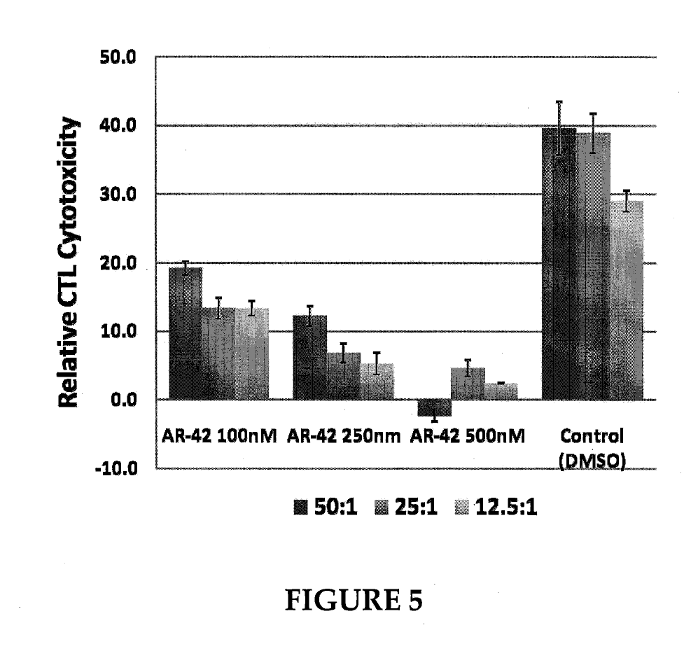

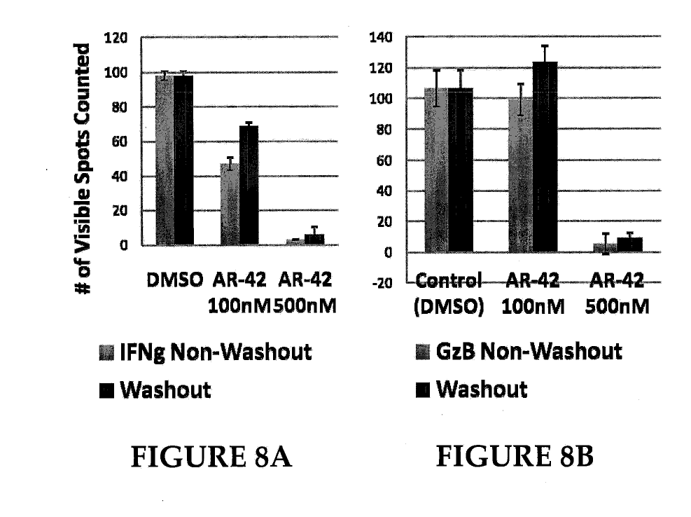

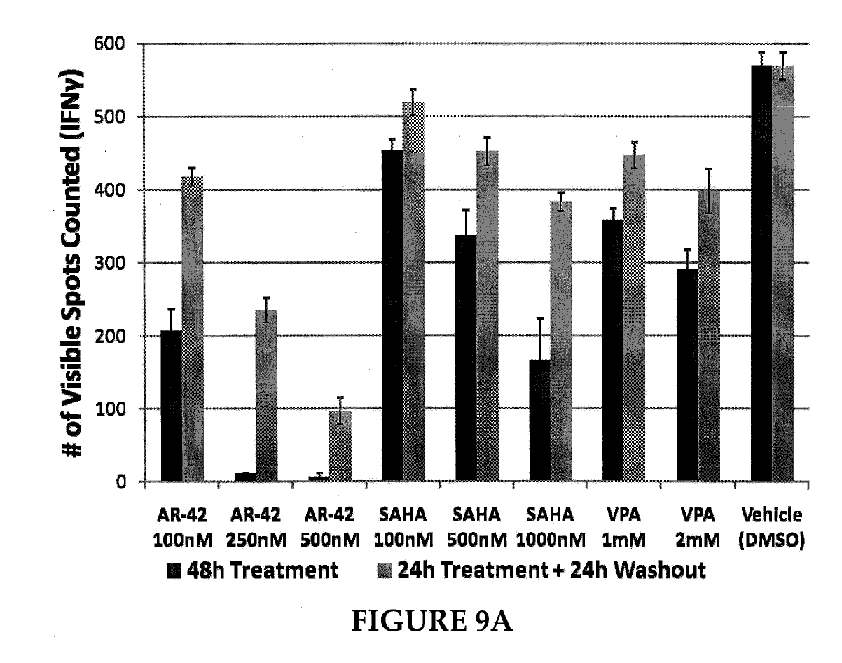

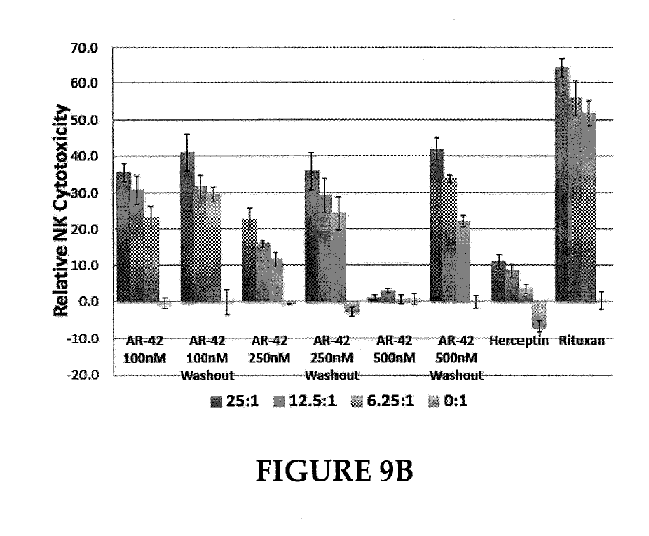

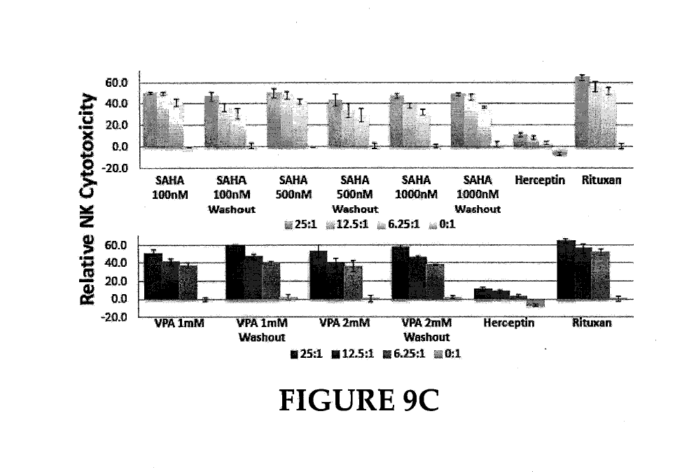

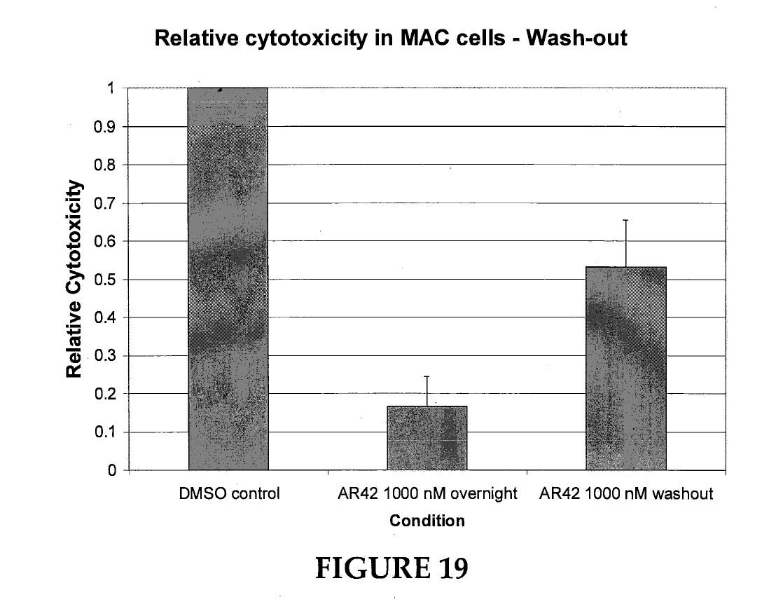

Treatment with AR-42 has a dose-dependent negative affect on the cytotoxic activity of both NK and T cell effectors. This can be seen, for example in the data shown in FIGS. 5, 9B, 16 and 18. However, as shown for example by the data in FIGS. 9B and 19, this affect is transient and reversible. The data in FIGS. 9B and 19 shows that upon washout of AR-42, the cytotoxic activity significantly increases. The data in FIG. 19 is only for 1000 nM AR-42 however, similar, if not even more significant, increases in cytotoxic activity would be expected at lower concentrations of AR-42 (as seen in FIG. 9B). Treatment with AR-42 also has a dose-dependent negative affect on granzyme B (GzB) and interferon-gamma (IFNγ) release by activated NK and T cells. This can be seen, for example, in the data of FIGS. 8A, 8B, 9A and 17. However, as show for example by the data in FIGS. 8A, 8B and 9A, this affect is transient and reversible; upon washout of AR-42, the granzyme B (GzB) and interferon-gamma (IFNγ) release by activated NK and T cells is seen to significantly increase. Thus, upon washout of AR-42, and the corresponding recovery of cytotoxic activity and IFNγ release from both NK and T cell effectors, the immune system will provide additional anti-tumor activity.

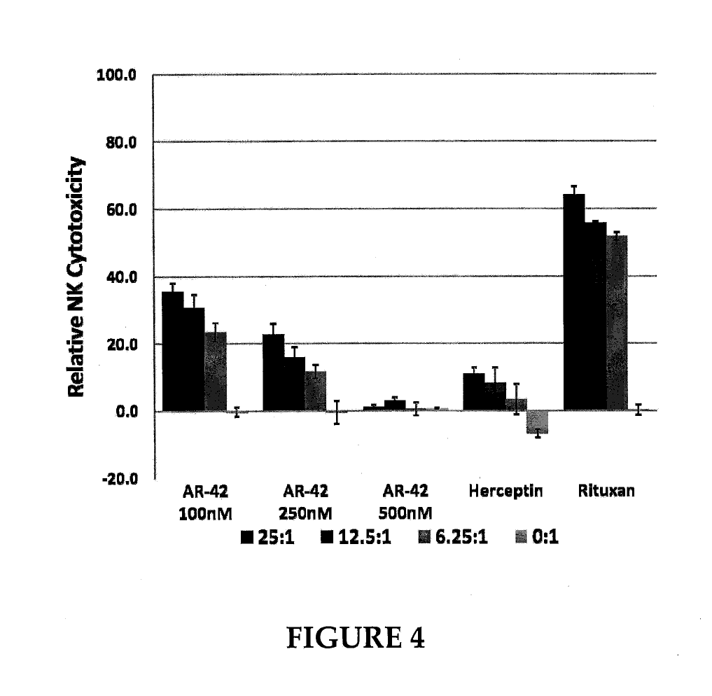

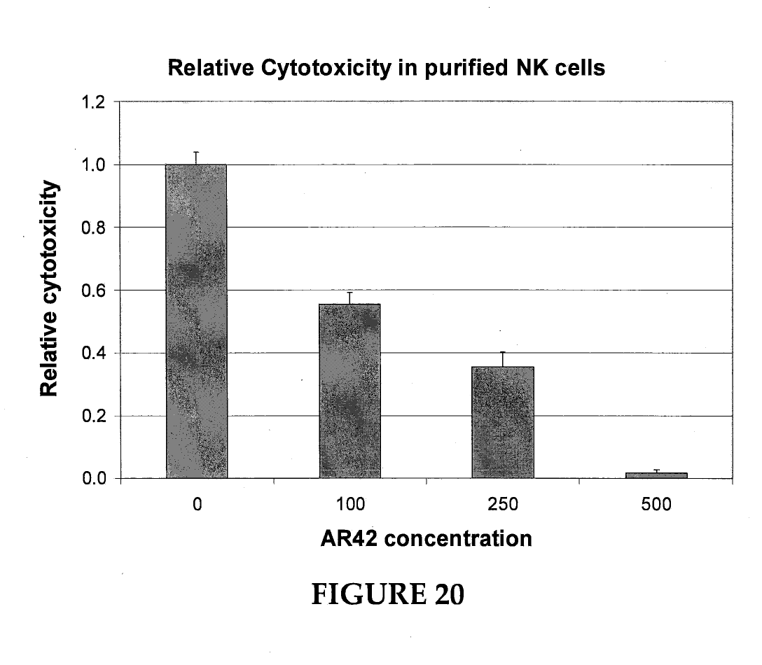

AR-42 also acts to enhance target expression of antigens that are then able to be targeted with antibodies. For example, treatment with AR-42 causes enhanced expression of antigens of EBV+ LCLs which, upon washout and associated restoration of immune function, allow for improved immune response against the tumor cells. The data shown in FIGS. 4 and 20 shows the effects of AR-42 on NK cell-driven antibody dependent cellular cytotoxicity. This mechanism provides yet another improvement in reduction in tumor cells by treatment with AR-42.

The observed inhibition of natural killer (NK) and T cell cytotoxicity occurs at concentrations (100-500 nM) below the range found to elicit anti tumor activity against Epstein-Barr Virus (EBV)-transformed lymphoblastoid cell lines (LCL). Overnight incubation of purified NK cells in low concentrations of AR-42 also inhibited antibody dependent cellular cytotoxicity (ADCC) against rituximab-coated EBV+ LCLs. Overnight incubation of effector and LCL target cells in low concentrations of AR-42 (100-500 nM) modulated expression of key regulatory proteins involved with cellular and adaptive immune responses (NKG2D, KIR, NKp46, NKp30, MHC I). AR-42 led to decreased granzyme B (GzB) and interferon-gamma (IFNγ) release by activated NK and T cells. Similar effects with IFNγ and GzB production were seen with the broad spectrum HDAC inhibitors suberoylanilide hydroxamic acid (SAHA) and valproate; however, NK cell cytotoxic potential remained unchanged with these two HDAC inhibitors. Washout of AR-42 from cell cultures led to significant recovery of cytotoxic activity and IFNγ release from both NK and T cell effectors show that the inhibitory effect is transient, fully reversible and comparable to vehicle (DMSO) treated effector cells. The differential effects observed with SAHA and VPA on cytotoxicity and cytokine release show that individual HDAC inhibitor drugs may exhibit distinct immune-modulatory profiles. The data shows that broad spectrum class I and II HDAC inhibitors suppress the adaptive and innate immune responses.

HDAC inhibitors, specifically AR-42, inhibit EBV+ LCL growth at concentrations at or above 250 nM, but may promote drug resistance at concentrations at above 750 nM. Further, the HDAC-I concentrations needed to modulate cellular immune function is less than the minimum inhibitory concentration of EBV+ LCL. AR-42 also inhibits cytotoxic activity of T cells against EBV+ LCL upon long-term exposure and NK cell-mediated ADCC against EBV+LCL upon short-term exposure. AR-42 and SAHA inhibits IFNγ release at low concentrations, but SAHA does not affect NK cytotoxic activity. AR-42 and SAHA also modulated some NK regulatory receptors. AR-42 and SAHA exert a transient and reversible effect, with the extent of the recovery corresponding with the treatment duration of AR-42 or SAHA.

Levels of innate immune function can be measured using various markers, such as, for example, NKG2D or KIR on NK cells. For tumor targets, expression of MHC Class I (KIR ligand) and Mic A/B (NKG2D ligand) may be monitored. Levels of T cell immune function can be measured by monitoring, for example, T cell activation markers (CD69, CD49a), memory status (CD45 RA, vs RO, CD27, CD29) and Treg (CD25, FoxP3, CD193, CD294, CD183).

Thus, AR-42 (or other HDAC inhibitor) can be used as a potent therapeutic for cancer treatment, in particular for treatment of lymphomas, use in post-transplant patients to treat transplant associated lymphoproliferative diseases, in the treatment of refractory Hodgkin's or non-Hodgkin's lymphoma, in the treatment of AIDS-associated lymphoma, or in treatment of autoimmune disorders. Immune function may be inhibited or reduced at low concentrations of AR-42 or HDAC inhibitors. In particular, low dose treatment with AR-42 can be used to maximize anti tumor activity while minimizing immune suppression. Formulations that comprise AR-42 (or another HDAC inhibitor) and a compound which limits the immune suppressive effects of AR-42 (or the other HDAC inhibitor) will be useful in the treatment of the various diseases discussed.

The HDAC inhibitor compounds used in the various aspects described herein can be administered orally, parenterally (IV, 1M, depot-1M, SQ, and depot-SQ), sublingually, intranasally (inhalation), intrathecally, topically, or rectally. Dosage forms known to those of skill in the art are suitable for delivery of the HDAC inhibitor compounds used in the various aspects described herein. The term “administer” refers to providing or prescribing the HDAC inhibitor to a mammal (e.g., human, dog, cat, cow, pig, sheep, etc.).

Compositions are provided that contain therapeutically effective amounts of the HDAC inhibitor compounds used in the various aspects described herein. The compounds can be formulated into suitable pharmaceutical preparations such as tablets, capsules, or elixirs for oral administration or in sterile solutions or suspensions for parenteral administration. The compounds described herein can be formulated into pharmaceutical compositions using techniques and procedures well known in the art.

The HDAC inhibitor compounds used in the various aspects described herein, or a physiologically acceptable salt or ester is compounded with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage in the range indicated is obtained. The compositions can be formulated in a unit dosage form, each dosage containing an amount of active substance sufficient to achieve a plasma concentration from about 100 nM to about 2 uM, and more specifically an amount sufficient to achieve a plasma concentration from about 100 nM to about 1000 nM, or about 250 nM to about 750 nM, or about 250 nM to about 1000 nM. An amount of active substance sufficient to achieve the desired plasma concentrations may be 0.1 mg to 100 mg; more preferably the amount of active substance sufficient to achieve the desired plasma concentrations may be 10 mg to 50 mg; even more preferably the amount of active substance sufficient to achieve the desired plasma concentrations may be 20 mg to 40 mg. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

In one aspect, the dose of the HDAC inhibitor is about 0.1 mg to about 100 mg, preferably 10 mg to about 50 mg, more preferably from about 20 mg to about 40 mg. These HDAC inhibitor dosages are also referred to herein as a “low dose” of the HDAC inhibitor.

In one aspect, pharmaceutical compositions comprising an HDAC inhibitor and a pharmaceutically acceptable carrier are provided. The HDAC inhibitor in these compositions is present in an amount sufficient to achieve a plasma concentration from about 100 nM to about 2 uM in a mammal after administration of the HDAC inhibitor to the mammal. Alternatively, the plasma concentration achieved is about 100 nM to about 1000 nM, 250 nM to about 750 nM, 100 nM, or 250 nM. In another aspect, an amount sufficient to achieve the plasma concentration is a low dose of the HDAC inhibitor.

In another aspect, the amount of an HDAC inhibitor sufficient to achieve the described plasma concentrations can be readily determined by one of skill in the art by administering a first dose of an HDAC inhibitor and monitoring the plasma concentration. Subsequent doses of the HDAC inhibitor can be adjusted to maintain the plasma concentration as described herein.

In another aspect, pharmaceutical compositions comprising an HDAC inhibitor and a pharmaceutically acceptable carrier are provided wherein the concentration of the HDAC inhibitor is sufficient to decrease the relative viability of lymphoblastoid cells by at least about 50 percent. In another aspect, an amount sufficient to decrease the relative viability of lymphoblastoid cells by at least about 50 percent is a low dose of the HDAC inhibitor.

In another aspect, pharmaceutical compositions comprising an HDAC inhibitor and a pharmaceutically acceptable carrier are provided wherein the concentration of the HDAC inhibitor is sufficient to decrease the proliferation of lymphoblastoid cells by at least about 60 percent. In another aspect, an amount sufficient to decrease the proliferation of lymphoblastoid cells by at least about 60 percent is a low dose of the HDAC inhibitor.

In another aspect, pharmaceutical compositions comprising an HDAC inhibitor and a pharmaceutically acceptable carrier are provided wherein the concentration of the HDAC inhibitor is sufficient to decrease the relative viability of peripheral blood mononuclear cells by less than about 50 percent or less than about 30 percent. In another aspect, an amount sufficient to decrease the relative viability of peripheral blood mononuclear cells by less than about 50 percent or less than about 30 percent is a low dose of the HDAC inhibitor.

In yet another aspect, the HDAC inhibitor is selected from the group consisting of SAHA, valproate, and AR 42 (N-hydroxy-4-(3-methyl-2-phenyl-butyrylamino)-benzamide). In yet another aspect, the HDAC inhibitor is AR 42.

In another aspect, a method of decreasing the relative viability of lymphoblastoid cells by at least about 50 percent is provided. The method includes administering an HDAC inhibitor to a mammal in an amount sufficient to achieve a plasma concentration of about 250 nM in the mammal. In another aspect, the relative viability of peripheral blood mononuclear cells is decreased by less than about 50 percent, or by less than about 30 percent. In another aspect, an amount sufficient to achieve a plasma concentration of about 250 nM in the mammal and to decrease the relative viability of peripheral blood mononuclear cells by less than about 50 percent or less than about 30 percent is a low dose of the HDAC inhibitor.

In another aspect, a method of decreasing the proliferation of lymphoblastoid cells by at least about 60 percent is provided. The method includes administering an HDAC inhibitor to a mammal in an amount sufficient to achieve a plasma concentration of about 100 nM in the mammal. In another aspect, the relative viability of peripheral blood mononuclear cells is decreased by less than about 50 percent, or by less than about 30 percent. In another aspect, an amount sufficient to achieve a plasma concentration of about 100 nM in the mammal and to decrease the relative viability of peripheral blood mononuclear cells by less than about 50 percent or less than about 30 percent is a low dose of the HDAC inhibitor.

In another aspect, a method of treating a lymphoproliferative disease comprising administering an HDAC inhibitor and a pharmaceutically acceptable carrier to a mammal in need of treatment is provided. The method includes administering the HDAC inhibitor in an amount sufficient to achieve a plasma concentration of about 100 nM to about 2 uM in the mammal. Alternatively, the plasma concentration achieved is about 100 nM to about 250 nM, about 250 nM to about 500 nM, about 500 nM to about 750 nM, or about 750 nM to about 1000 nM. In another aspect, the relative viability of peripheral blood mononuclear cells is decreased by less than about 50 percent, or by less than about 30 percent. In another aspect, an amount sufficient to achieve a plasma concentration of about 100 nM to about 2 uM in the mammal is a low dose of the HDAC inhibitor.

In another aspect, the method further includes terminating or reducing administration of the HDAC inhibitor to the mammal wherein the plasma concentration of the HDAC inhibitor is reduced. In this method, interferon-gamma (IFNγ) release levels increase by at least 50 percent as compared to interferon-gamma (IFNγ) release levels during administration of the HDAC inhibitor about 24 hours after terminating or reducing the administration of the HDAC inhibitor. In this method, granzyme B (GzB) release levels increase by at least 25 percent as compared to granzyme B (GzB) release levels during administration of the HDAC inhibitor about 24 hours after terminating or reducing administration of the HDAC inhibitor. In this method, relative cytotoxicity levels increase by at least 50 percent as compared to relative cytotoxicity levels during administration of the HDAC inhibitor about 24 hours after terminating or reducing administration.

In another aspect, the method may include administering the HDAC inhibitor in an amount sufficient to achieve a plasma concentration of about 100 nM to about 2 uM in the mammal only on alternating days, such that washout occurs between each administration of the HDAC inhibitor.

To prepare compositions, one or more HDAC inhibitor compounds used in the various aspects described herein are mixed with a suitable pharmaceutically acceptable carrier. Upon mixing or addition of the compound(s), the resulting mixture may be a solution, suspension, emulsion, or the like. Liposomal suspensions may also be used as pharmaceutically acceptable carriers. These may be prepared according to methods known to those skilled in the art. The form of the resulting mixture depends upon a number of factors, including the intended mode of administration and the solubility of the compound in the selected carrier or vehicle. The effective concentration is sufficient for lessening or ameliorating at least one symptom of the disease, disorder, or condition treated and may be empirically determined.

Pharmaceutical carriers or vehicles suitable for administration of the HDAC inhibitor compounds used in the various aspects described herein include any such carriers suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

Where the compounds exhibit insufficient solubility, methods for solubilizing may be used. Such methods are known and include, but are not limited to, using co-solvents such as dimethylsulfoxide (DMSO), using surfactants such as TWEEN, and dissolution in aqueous sodium bicarbonate. Derivatives of the compounds, such as salts or prodrugs, may also be used in formulating effective pharmaceutical compositions.

The concentration of the compound is effective for delivery of an amount upon administration that lessens or ameliorates at least one symptom of the disorder for which the compound is administered. Typically, the compositions are formulated for single dosage administration.

The HDAC inhibitor compounds used in the various aspects described herein may be prepared with carriers that protect them against rapid elimination from the body, such as time-release formulations or coatings. Such carriers include controlled release formulations, such as, but not limited to, microencapsulated delivery systems. The active compound can be included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo model systems for the treated disorder.

The HDAC inhibitor compounds used in the various aspects described herein can be enclosed in multiple or single dose containers. The enclosed compounds and compositions can be provided in kits, for example, including component parts that can be assembled for use. For example, an HDAC inhibitor compound in lyophilized form and a suitable diluent may be provided as separated components for combination prior to use. A kit may include an HDAC inhibitor compound and a second therapeutic agent for co-administration. The HDAC inhibitor compound and second therapeutic agent may be provided as separate component parts. A kit may include a plurality of containers, each container holding one or more unit dose of the HDAC inhibitor compound. The containers can be adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The concentration of active inventive compound in the drug composition will depend on absorption, inactivation, and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual's need and the professional judgment of the person administering or supervising the administration of the compositions, and that the concentration ranges set forth herein are exemplary only and are not intended to limit the scope or practice of the claimed compositions and methods.

If oral administration is desired, the compound can be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a glidant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed with other active materials that do not impair the desired action, or with materials that supplement the desired action. The HDAC inhibitor compounds used in the various aspects described herein can be used, for example, in combination with an antitumor agent, a hormone, a steroid, or a retinoid. The antitumor agent may be one of numerous chemotherapy agents such as an alkylating agent, an antimetabolite, a hormonal agent, an antibiotic, colchicine, a vinca alkaloid, L-asparaginase, procarbazine, hydroxyurea, mitotane, nitrosoureas or an imidazole carboxamide. Suitable agents include those agents which promote depolarization of tubulin. Examples include colchicine and vinca alkaloids, including vinblastine and vincristine.

Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerin, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include, but are not limited to, physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known in the art.

The HDAC inhibitor compounds used in the various aspects described herein may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

The HDAC inhibitor compounds used in the various aspects described herein may be administered enterally or parenterally. When administered orally, these compounds can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, they can be of the sustained release type so that the compounds employed in the methods of the invention need to be administered only once or twice daily.

The oral dosage forms can be administered to the patient 1, 2, 3, or 4 times daily. The HDAC inhibitor compounds used in the various aspects described herein can be administered either three or fewer times, or even once or twice daily. Further, the oral dosage form may be administered to the patient for a period of for example 7, 10, 14 or 21 days and then treatment terminated or reduced for a period of 7, 10, 14 or 21 days (the same or different than the period of treatment) to allow washout of the HDAC inhibitor (e.g., reduction of the plasma concentration of the HDAC inhibitor in the mammal receiving treatment). Alternatively, the oral dosage form may be administered to the patient on alternating days (or three times per week). In this aspect, washout of the HDAC inhibitor may occur between each administration of the oral dosage form.

Hence, the HDAC inhibitor compounds used in the various aspects described herein be administered in oral dosage form. Whatever oral dosage form is used, they can be designed so as to protect the compounds employed from the acidic environment of the stomach. Enteric coated tablets are well known to those skilled in the art. In addition, capsules filled with small spheres each coated to protect from the acidic stomach, are also well known to those skilled in the art.

The HDAC inhibitor compounds used in the various aspects described herein may also be advantageously delivered in a nanocrystal dispersion formulations. Preparation of such formulations is described, for example, in U.S. Pat. No. 5,145,684, the entire contents of which is incorporated by reference. Nanocrystalline dispersions of HIV protease inhibitors and their method of use are described in U.S. Pat. No. 6,045,829, the entire contents of which is incorporated by reference. The nanocrystalline formulations typically afford greater bioavailability of drug compounds.

The terms “therapeutically effective amount” and “therapeutically effective period of time” are used to denote treatments at dosages and for periods of time effective to reduce neoplastic cell growth. As noted above, such administration can be parenteral, oral, sublingual, transdermal, topical, intranasal, or intrarectal. When administered systemically, the therapeutic composition can be administered at a sufficient dosage to attain a blood level of the HDAC inhibitor compound of from about 100 nM to about 2 uM, and more specifically from about 250 nM to about 1000 nM or from about 250 nM to about 750 nM. One of skill in the art will appreciate that while a patient may be started at one dose, that dose may be varied overtime as the patient's condition changes.

It should be apparent to one skilled in the art that the exact dosage and frequency of administration will depend on the particular compounds employed administered in the methods of the various aspects, the particular condition being treated, the severity of the condition being treated, the age, weight, general physical condition of the particular patient, and other medication the individual may be taking as is well known to administering physicians who are skilled in this art.

### EXPERIMENTAL EXAMPLES

### Example 1

The effects of HDAC inhibition on the proliferation and survival of EBV transformed B cells were studied in vitro by culturing EBV+ transformed B lymphoblastoid cell lines (EBV+ LCL) in AR-42, SAHA, or valproate-containing media. In particular, EBV+ LCLs were cultured in media that contained: 0, 100, 250, 500, 750, & 1000 nM AR-42; 0, 250, 500, 1000, 2000, 4000 nM SAHA; 0, 0.5, 1, 2, 3, 4 mM valproate for 4 days. Cell viability was then analyzed every 24 hours by annexin/PI staining and flow cytometry.

As seen in FIG. 3A, B cell populations that were cultured in 250-1000 nM AR-42 concentrations experienced significant cell death by day 2 (87, 75, 62, & 54% viability, respectively). However, the B cell population that was cultured in 750 and 1000 nM AR-42 actually displayed higher viability by day 4 than the day 2 populations in the same culture. Cell viability was analyzed by annexin/PI staining and flow cytometry.

As seen in FIG. 3B, B cell populations cultured in 250-2000 nM SAHA resulted in little cell death during the 4 day incubation. Significant B cell death was observed in the 4000 nM SAHA condition by day 4 (69% viability). Cell viability was analyzed by annexin/PI staining and flow cytometry.

As seen in FIG. 3C, Class I-specific HDAC-I valproate resulted in some B cell death by day 3 at 2.0-4.0 mM (83, 73, & 63% viability, respectively). By day 4, the 4 mM condition's viability recovered (71%) in a fashion similar to AR-42 at higher doses. Cell viability was analyzed by annexin/PI staining and flow cytometry.

### Example 1A

The effects of AR-42 on the viability and proliferation of lymphoblastoid cell lines was determined. LCLs (C7M3, DC9) were cultured in media that contained 0, 100, 250, 500, & 1000 nM AR-42 for 4 days. Cell viability was analyzed by annexin/PI staining and flow cytometry. Cell counts were measured at day 5. The results are seen in FIGS. 14A and 14B. In FIGS. 14A and 14B, the standard deviation represents the results from triplicate experiments.

### Example 2

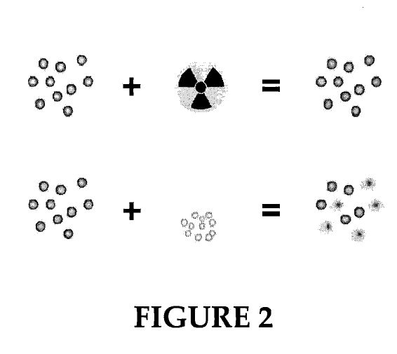

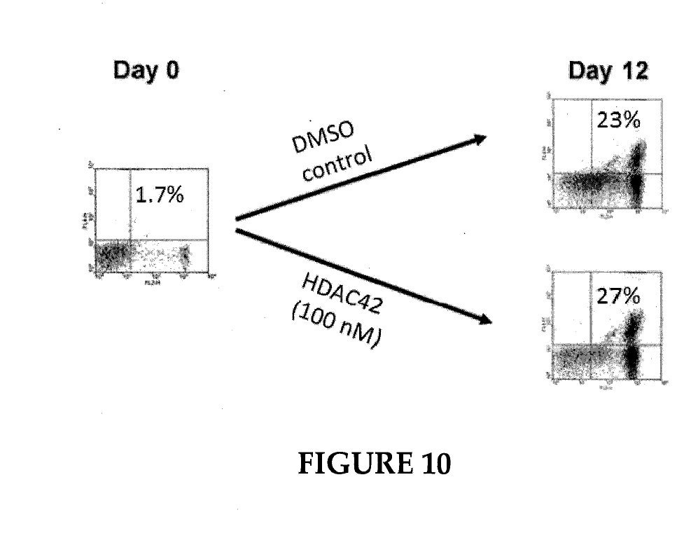

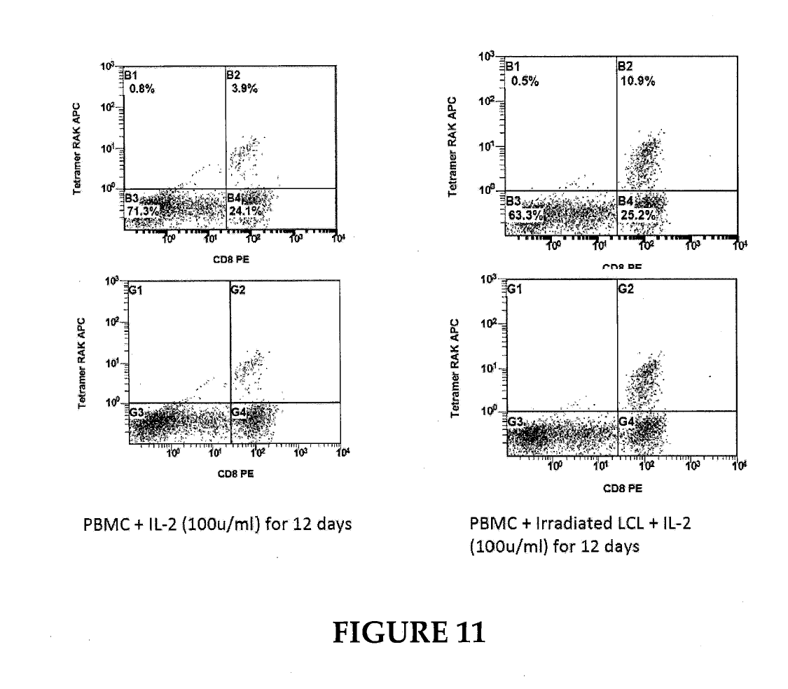

The effects of AR-42 on EBV-specific cytotoxic T cell (CTL) cytotoxicity were assessed in vitro. Peripheral blood mononuclear cells (PBMC) and autologous EBV+ LCL were co-culturing in media containing IL-2 (50 μM) and either DSMO (control) or AR-42 (100 nM). EBV-specific CTLs were identified with anti-CD8 mAb and HLAB8-RAK tetramer, upon a 12 day expansion, the results of which are shown in FIG. 10. FIG. 11 shows flow data from another expansion comparing PBMCs not-stimulated with LCLs to PBMCs stimulated with LCLs. At day 13, the EBV-specific CTLs were incubated overnight in 100, 250, & 500 nM AR-42+IL-2 (50 μM). The next day, a four hour ⁵¹Cr release assay was performed on each incubated sample in order to access the effects of AR-42 on EBV-specific cytotoxic T cell (CTL) cytotoxicity. This is shown schematically in FIG. 2, which illustrates autologous target EBV+ LCLs are labeled with ⁵¹Cr, which is actively internalized by each cell via pumps in its membrane; effector CTLs are counted and plated at desired effector-target ratios; labeled targets are incubated with effectors for four hours; and lysed targets release intracellular ⁵¹Cr into the supernatant, which is harvested and analyzed for radioactivity using a gamma counter. Effector-target ratios of 50:1, 25:1, and 12.5:1 were used. As seen in FIG. 5, the CTLs treated overnight with 100 and 250 nM AR-42 indicated a reduced capability to kill target B cells at the highest effector-target ratio (19 and 12%, respectively). Treatment with 500 nM AR-42 limited CTL cytotoxicity significantly (−2%).

### Example 2A

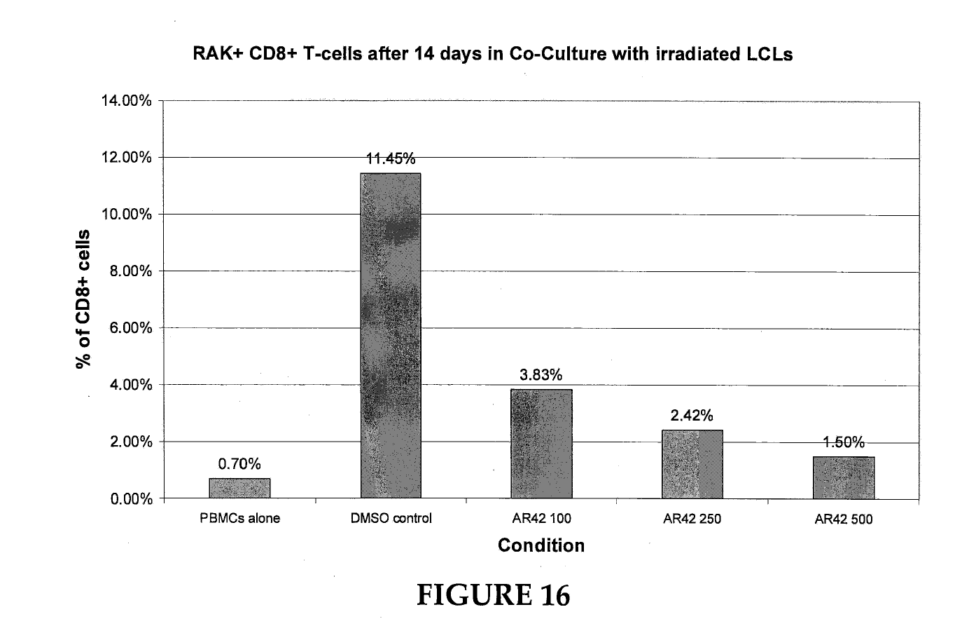

The effect of AR-42 on EBV-specific T-cell expansion was determined. Irradiated LCLs were added to purified PBMCs and maintained in co-culture for two weeks with AR-42 (100 nM, 250 nM, 500 nM). After two weeks, the EBV-specific T-cell expansion was measured using the RAK tetramer. This tetramer contains the RAKFQLLL peptide which binds the HLA-B8 MHC type I epitope, and can be used to identify EBV-specific (BZLF1 specific) CD8+ T-cells. FIG. 16 shows the results of a representative experiment. As can be seen in FIG. 16, AR-42 decreases EBV-specific T-cell expansion in a dose dependent manner.

### Example 3

The effects of AR-42 on NK cell-driven antibody dependent cellular cytotoxicity (ADCC) were assessed in vitro. NK cells were treated overnight with AR-42 (100 nM, 250 nM, 500 nM) or Rituximab (anti-CD20) or Herceptin (anti-erbB2). Rituximab (anti-CD20) was the antibody of choice for the assay because of its wide clinical use and proven efficacy against B cell lymphomas that express CD20 antigen. Herceptin (anti-erbB2), a commonly used breast cancer drug, was used as a negative control. The effect of AR-42 on NK cell-driven antibody dependent cellular cytotoxicity (ADCC) was assessed using a four hour ⁵¹Cr release assay. The four hour ⁵¹Cr release assay was performed as described with respect to Example 2A, except that effector-target ratios of 25:1, 12.5:1, & 6.25:1 were used.

As seen in FIG. 4, NK cells treated with 100-250 nM AR-42 displayed a reduced capability to kill target B cells in combination with rituximab at the highest effector-target ratio (36 and 23%, respectively) as compared to the control at the max E:T ratio (64%). Treatment with 500 nM AR-42 limited NK cytotoxicity significantly (1.1%). NK cells that were not treated with AR-42 displayed high relative ADCC at 25:1, 12.5:1, & 6.25:1 effector-target ratios in combination with rituximab (e.g., Rituxan®) (64, 56, & 52%, respectively).

### Example 4

The production of proteins associated with the T cell cytotoxic response (interferon-γ, granzyme B) was determined in vitro. Activated EBV-specific T cells were initially treated for 4 days (T) with 100 nM or 500 nM AR-42 and further stimulated with EBV+ LCLs (effector-target ratio: 1:1) in an ELISPOT assay to determine IFNγ and GzB secretion potentials. Release of interferon-gamma (IFNγ) or granzyme B (GzB) was visualized using monoclonal IFNγ/GzB antibodies with BCIP/NBT substrate solution. Results were quantified by an enzyme-linked immunosorbent spot assay (ELISPOT) analyzer.

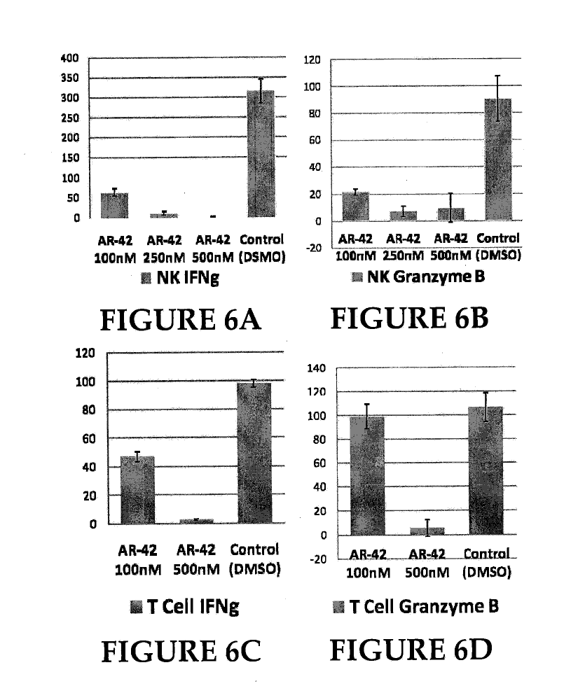

As seen in FIGS. 6C and 6D, T cells treated with 100 and 500 nM AR-42 resulted in decreased IFNγ and GzB secretion (47, 3 spots and 99, 5 spots, respectively) in a dose-dependent manner compared to the untreated control (98 and 107 spots, respectively).

### Example 5

Purified NK cells were initially treated overnight (NK) with 100 nM, 250 nM or 500 nM AR-42. The NK cells were further stimulated using inflammatory cytokines cytokines IL-2 (50 pM) and IL-12 (10 ng/ml) in an ELISPOT assay to determine IFNγ and GzB secretion potentials. Results were quantified by an ELISPOT analyzer.

As seen in FIGS. 6A and 6B, NK cells treated with 100, 250, and 500 nM AR-42 produced significantly less IFNγ and GzB (63, 10, 1 spots and 22, 7, 10 spots, respectively) in a dose-dependent manner compared to the untreated control (314 and 91 spots, respectively).

### Example 6

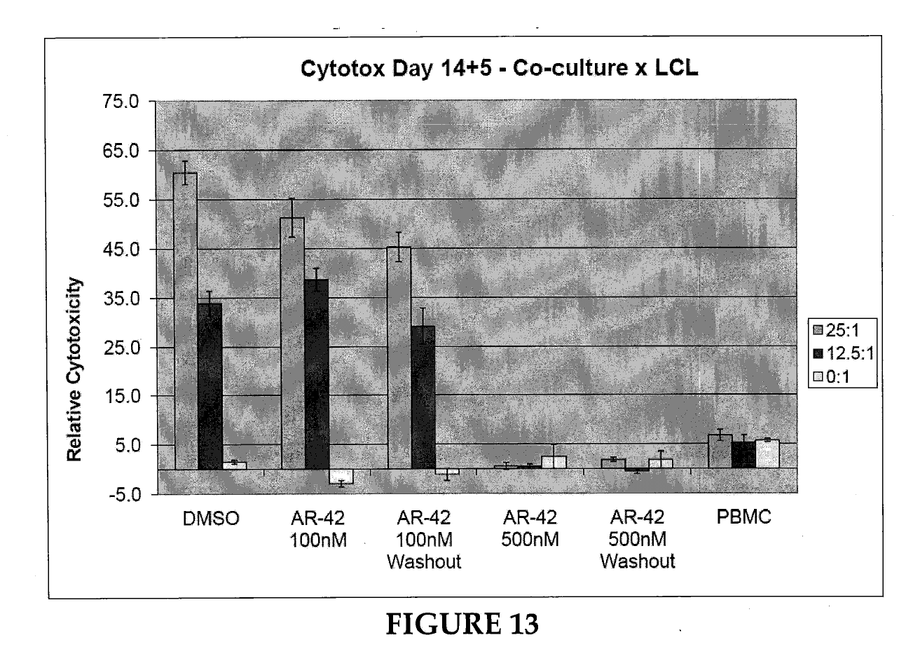

Activated EBV-specific T cells expanded in the presence of AR-42 were further incubated in media with and without AR-42. After 5 days, the T cells were stimulated with EBV+ LCLs in an ELISPOT assay to determine IFNγ and GzB secretion potentials. As seen in FIGS. 8A and 8B, modest recovery of IFNγ and GzB production was observed in the washout samples at 100 nM AR-42. The data shows that long-term exposure to 500 nM AR-42 results in eventual loss of cytotoxic function of activated T cells. This was also reflected in the corresponding four hour ⁵¹Cr release assay, as shown in FIG. 13.

### Example 7

Purified NK cells were treated with 100 nM, 250 nM, or 500 nM AR-42; 100 nM, 500 nM or 1000 nM SAHA; or 1 mM or 2 mM valproate for either 48 hours continuously or through a 24 hour treatment+24 hour washout regimen. Purified NK cells in DMSO were used as a control. The cells were subsequently analyzed in an IFNγ ELISPOT assay or a ⁵¹Cr release cytotoxicity assay.

As seen in FIG. 9A, compared to the control (569 spots), the 100, 250, and 500 nM AR-42-treated NK cells resulted in fewer spots (207, 10, and 6 spots) indicative of substantially decreased IFNγ secretion from NK cells. The corresponding AR-42 washout conditions revealed significant recovery of NK cytotoxic function (418, 235, 96 spots, respectively). A similar inhibitory effect and functional recovery was present in the 100, 500, and 1000 nM SAHA-treated NK cells (452, 366, 166 spots). Valproate did not inhibit NK IFNγ secretion to the same degree as AR-42 or SAHA, despite its high concentrations of 1 and 2 mM (446, 238 spots).

As seen in FIG. 9B, the cytotoxic activity of 100, 250, and 500 nM AR-42-treated NK cells followed a dose-dependent trend of inhibition (36, 23, & 1%, respectively) as compared to the control at the max E:T ratio (64%) (also discussed with respect to FIG. 4 above). However, the corresponding AR-42 washout conditions recovered the NK cytotoxic activity to a near uniform level across the three treated cell lines (41, 36, & 42%). The data shows that short-term exposure to AR-42 permits significant recovery of NK cytotoxic activity. As shown in FIG. 9C, no significant ADCC inhibition was observed in the SAHA and valproate conditions.

### Example 8

The mechanism by which HDAC inhibition affects NK cell-driven ADCC was investigated in vitro. NK cells and EBV+ LCLs were incubated separately in AR-42 (100 nM, 500 nM), SAHA (100 nM, 1000 nM), or valproate (1, mM, 2 mM) and control (DMSO) for 24 hours. Viable cells were then collected and stained with monoclonal antibodies specific for cell surface proteins with known stimulatory (NKG2D, FcγRIII) or inhibitory (KIR) activity. Protein expression was then quantified via flow cytometry.

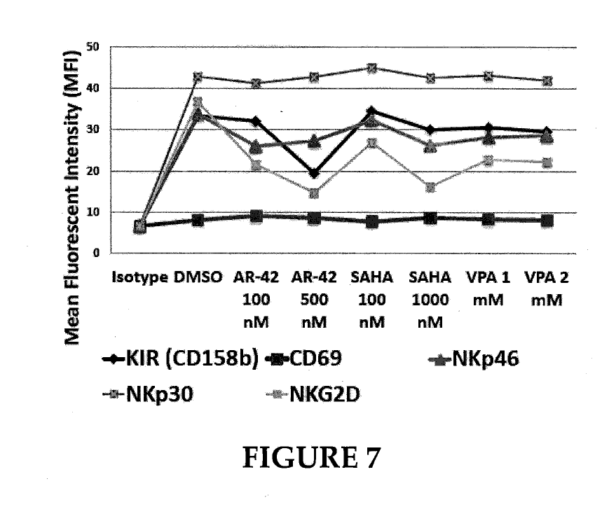

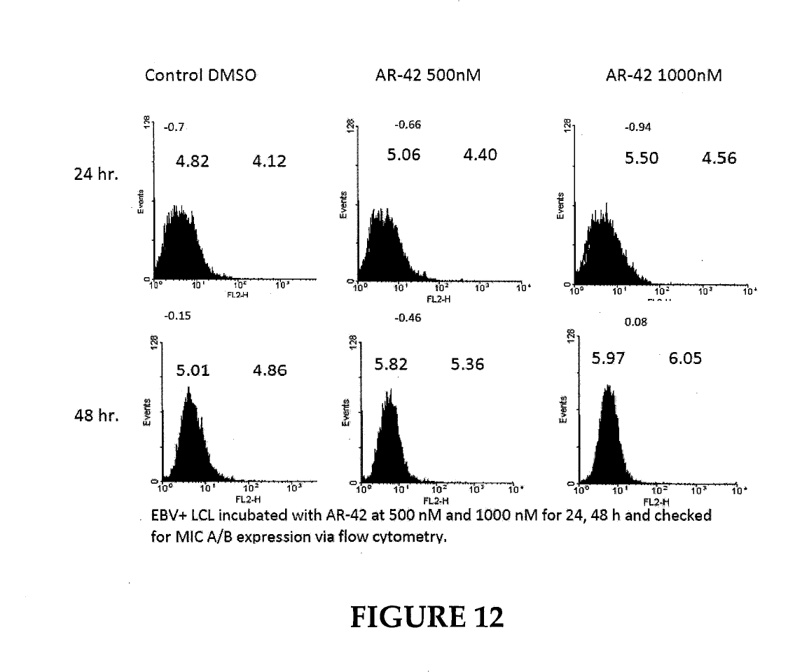

As seen in FIG. 7, AR-42 and SAHA both down modulate expression of the activating NK cell receptor NKG2D on purified CD56+ NK cells. AR-42 down modulates the inhibitory receptor KIR on NK cells, especially at 500 nM. AR-42 also down modulates MHC Class I expression (KIR ligand) on EBV+ LCLs at higher concentrations while Mic A/B (NKG2D ligand) was unaffected, as shown in FIG. 12. Little change was observed with valproate.

### Example 9

The effect of AR-42 on the viability of peripheral blood mononuclear cells (PBMCs) was determined. PBMCs were isolated from three random donor leukopaks and incubated with AR-42 (100 nM, 250 nM, 500 nM, 1000 nM) for 2 and 4 days. Viability was assessed by PI staining using flow cytometry. All results were normalized relative to untreated DMSO controls. As can be seen in FIG. 15, AR-42 decreases the viability of PBMCs in a dose dependent manner and at concentrations above 250 nM, there is a continued decrease in viability from 2 to 4 days. The standard deviations in FIG. 15 represent the mean of the three leukopaks.

### Example 10

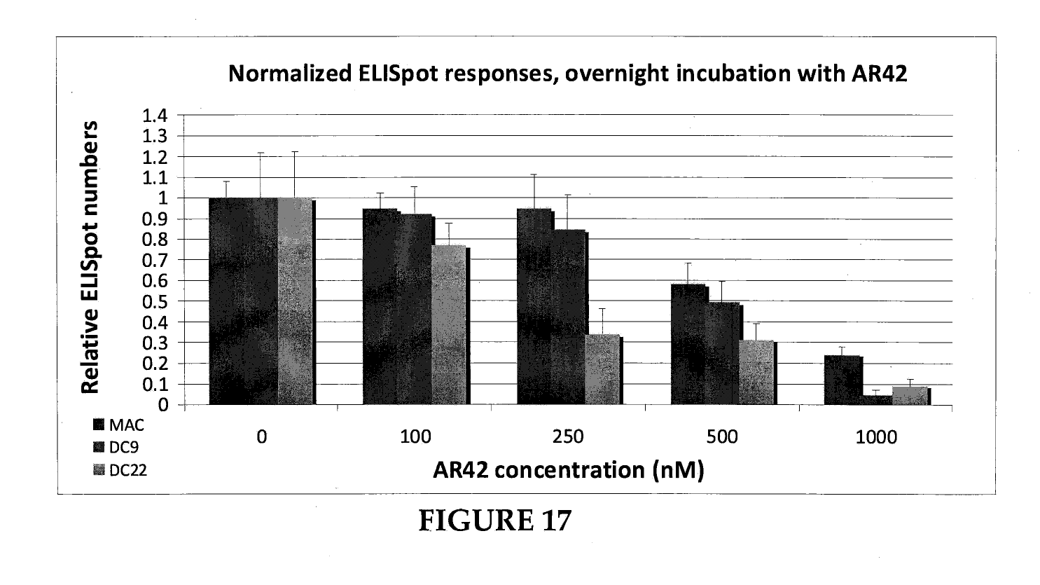

The effect of AR-42 on the release of interferon gamma was determined. Irradiated LCLs were added to purified PBMCs and maintained in co-culture (without AR-42) for two weeks. The co-cultured PBMCs were then incubated overnight with AR-42 (0 nM, 100 nM, 250 nM, 500 nM, 1000 nM) and assayed for interferon-gamma release by ELISPOT (10,000 effector cells per well: 1,000 fresh LCLs). The results are shown in FIG. 17 for three separate PBMC donors and are normalized to untreated DMSO controls. The standard deviations represent the data from four different ELISPOT wells.

### Example 11

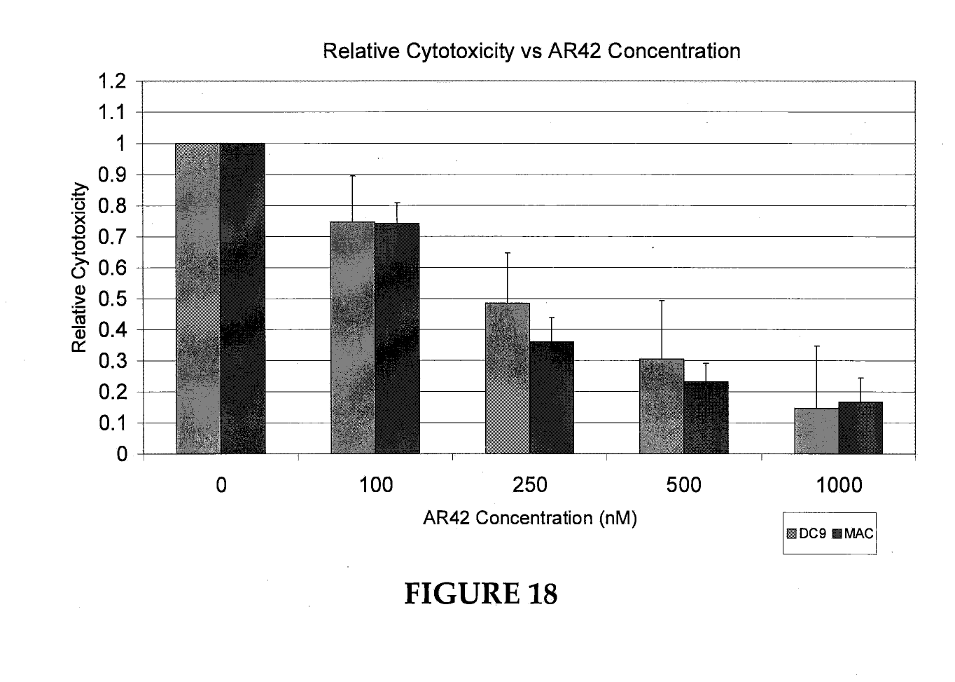

The effect of AR-42 on cellular cytoxic immune response was determined. Irradiated LCLs were added to purified PBMCs and maintained in co-culture for two weeks (without AR-42). Co-cultured PBMCs were then incubated overnight with AR-42 (0 nM, 100 nM, 250 nM, 500 nM, 1000 nM) and added (in a ratio of 25:1) to fresh LCLs (stained with CFSE). After 4 hours, LCL viability was measured by 7-AAD staining with flow cytometry. Cytotoxicity is normalized to untreated DMSO controls (minus background). The results shown in FIG. 18 are from two separate PBMC donors and the standard deviation reflects the results of three separate experiments.

### Example 12

It was determined whether the effects of AR-42 on cellular cytoxic immune response is reversible upon washout. Irradiated LCLs were added to purified PBMCs and maintained in co-culture for two weeks (without AR-42). Co-cultured PBMCs were then incubated overnight with AR-42 (1000 nM). In some samples, the AR-42 was washed out. The cells were then re-suspended in fresh media for 24 hours. Co-cultured PBMCs were then added (in a ratio of 25:1) to fresh LCLs (stained with CFSE). After 4 hours, LCL viability was measured by 7-AAD staining with flow cytometry. Cytotoxicity is normalized to untreated DMSO controls (minus background). The results shown in FIG. 19 are from a single PBMC donors and the standard deviation reflects the results of three separate experiments. As can be seen in FIG. 19, a partial recovery of cellular cytoxic immune response is seen in the AR-42 washout sample.

### Example 13

The effect of AR-42 on antibody dependent cellular cytotoxic immune response were determined. NK cells were added to autologous LCLs and incubated with rituximab. Cells were then incubated in culture for 24 hours with AR-42 (0 nM, 100 nM, 250 nM, 500 nM). Standard 4-hours chromium release assays were used to assess the antibody dependent cellular cytotoxic immune responses. The results, shown in FIG. 20, represent one donor and are done in quadruplicate.

### Example 14

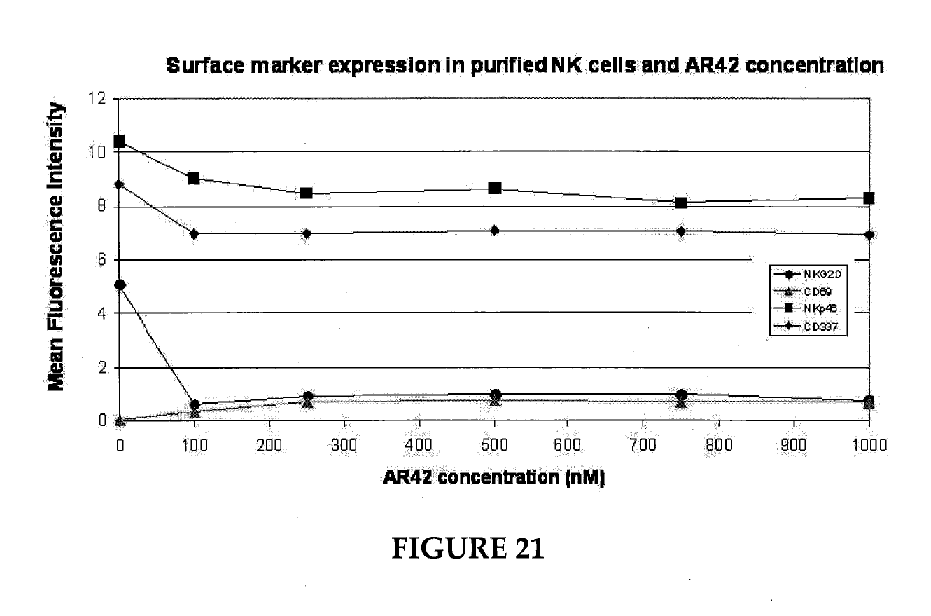

The effect of AR-42 on surface marker expression in NK cells was determined. NK cells were exposed to AR-42 (0 nM, 100 nM, 250 nM, 500 nM, 750 nM, 1000 nM). Alterations in surface marker (NKG2D, CD69, NKp46, CD337) expression in viable cells were evaluated the next day using flow cytometry. Results are shown in FIG. 21.

Various observations and conclusions can be made based on the experimental data. HDAC inhibitors affect EBV+ LCL growth in a dose dependent fashion. AR-42 decreases viability and proliferation of LCLs and to a lesser extent viability of PBMCs. Drug resistance mechanisms may develop at doses of AR-42 at or above 750 nM. AR-42 inhibits CTL direct cytotoxicity against autologous EBV+ LCL targets, NK cell-driven ADCC against autologous EBV+ LCL targets and NK and T cell interferon-γ and granzyme B release in a dose-dependent manner; however all of these effects are transient and reversible. HDAC inhibitors modulate activating and inhibitory markers on NK cells.

1. A pharmaceutical composition comprising an HDAC inhibitor and a pharmaceutically acceptable carrier, wherein the HDAC inhibitor is present in an amount sufficient to achieve a plasma concentration from about 100 nM to about 2 uM in a mammal after administration of the HDAC inhibitor to the mammal.

2. The pharmaceutical composition of claim 1, wherein the HDAC inhibitor is N-hydroxy-4-(3-methyl-2-phenyl-butyrylamino)-benzamide.

3. The pharmaceutical composition of claim 1, wherein the plasma concentration is from about 100 nM to about 1000 nM.

4. The pharmaceutical composition of claim 1, wherein the plasma concentration achieved is from about 250 nM to about 750 nM.

5. The pharmaceutical composition of claim 1, wherein the pharmaceutical composition comprises about 20 mg to about 40 mg of the HDAC inhibitor.

6. A pharmaceutical composition comprising an HDAC inhibitor and a pharmaceutically acceptable carrier, wherein the HDAC inhibitor is present in an amount from about 0.1 mg to about 100 mg.

7. The pharmaceutical composition of claim 6, wherein the HDAC inhibitor is present in an amount from about 10 mg to about 50 mg.

8. The pharmaceutical composition of claim 6, wherein the HDAC inhibitor is present in an amount from about 20 mg to about 40 mg.

9. A pharmaceutical composition comprising an HDAC inhibitor and a pharmaceutically acceptable carrier, wherein the concentration of the HDAC inhibitor is sufficient to decrease the relative viability of lymphoblastoid cells by at least about 50 percent.

10. The pharmaceutical composition of claim 9, wherein the HDAC inhibitor is N-hydroxy-4-(3-methyl-2-phenyl-butyrylamino)-benzamide.

11. The pharmaceutical composition of claim 9, wherein the HDAC inhibitor is present in an amount sufficient to achieve a plasma concentration of about 250 nM in a mammal after administration of the HDAC inhibitor to the mammal.

12. The pharmaceutical composition of claim 9, wherein the pharmaceutical composition comprises about 20 mg to about 40 mg of the HDAC inhibitor.

13. A pharmaceutical composition comprising an HDAC inhibitor and a pharmaceutically acceptable carrier, wherein the concentration of the HDAC inhibitor is sufficient to decrease the proliferation of lymphoblastoid cells by at least about 60 percent.

14. The pharmaceutical composition of claim 13, wherein the HDAC inhibitor is N-hydroxy-4-(3-methyl-2-phenyl-butyrylamino)-benzamide.

15. The pharmaceutical composition of claim 13, wherein the HDAC inhibitor is present in an amount sufficient to achieve a plasma concentration of about 100 nM in a mammal after administration of the HDAC inhibitor to the mammal.

16. The pharmaceutical composition of claim 13, wherein the pharmaceutical composition comprises about 20 mg to about 40 mg of the HDAC inhibitor.

17. A pharmaceutical composition comprising an HDAC inhibitor and a pharmaceutically acceptable carrier, wherein the concentration of the HDAC inhibitor is sufficient to decrease the relative viability of peripheral blood mononuclear cells by less than about 50 percent.

18. The pharmaceutical composition of claim 17, the concentration of the HDAC inhibitor is sufficient to decrease the relative viability of peripheral blood mononuclear cells by less than about 30 percent.

19. The pharmaceutical composition of claim 17, wherein the pharmaceutical composition comprises about 20 mg to about 40 mg of the HDAC inhibitor.

20. A method of decreasing the relative viability of lymphoblastoid cells by at least about 50 percent comprising administering an HDAC inhibitor to a mammal in an amount sufficient to achieve a plasma concentration of about 250 nM in the mammal.

21. The method of claim 20, wherein relative viability of peripheral blood mononuclear cells is decreased by less than about 50 percent.

22. The method of claim 20, wherein relative viability of peripheral blood mononuclear cells is decreased by less than about 30 percent.

23. The method of claim 20, wherein the HDAC inhibitor is N-hydroxy-4-(3-methyl-2-phenyl-butyrylamino)-benzamide.

24. A method of decreasing the proliferation of lymphoblastoid cells' by at least about 60 percent comprising administering an HDAC inhibitor to a mammal in an amount sufficient to achieve a plasma concentration of about 100 nM in the mammal.

25. The method of claim 24, wherein the relative viability of peripheral blood mononuclear cells is decreased by less than about 50 percent.

26. The method of claim 24, wherein the relative viability of peripheral blood mononuclear cells is decreased by less than about 30 percent.

27. The method of claim 24, wherein the HDAC inhibitor is N-hydroxy-4-(3-methyl-2-phenyl-butyrylamino)-benzamide.

28. A method of treating a lymphoproliferative disease comprising administering an HDAC inhibitor and a pharmaceutically acceptable carrier to a mammal in need of treatment, wherein the HDAC inhibitor is administered in an amount sufficient to achieve a plasma concentration of about 100 nM to about 2 uM in the mammal.

29. The method of claim 28, wherein the plasma concentration is about 100 nM to about 250 nM.

30. The method of claim 28, wherein the plasma concentration is about 250 nM to about 500 nM.

31. The method of claim 28, wherein the plasma concentration is about 500 nM to about 750 nM.

32. The method of claim 28, wherein the plasma concentration is about 750 nM to about 1000 nM.

33. The method of claim 28, wherein the relative viability of peripheral blood mononuclear cells in the mammal is decreased by less than about 50 percent.

34. The method of claim 28, wherein the relative viability of peripheral blood mononuclear cells in the mammal is decreased by less than about 30 percent.

35. The method of claim 28, further comprising terminating or reducing administration of the HDAC inhibitor to the mammal wherein the plasma concentration of the HDAC inhibitor is reduced.

36. The method of claim 35, wherein about 24 hours after terminating or reducing the administration of the HDAC inhibitor, interferon-gamma (IFNγ) release levels increase by at least 50 percent as compared to interferon-gamma (IFNγ) release levels during administration of the HDAC inhibitor.

37. The method of claim 35, wherein about 24 hours after terminating or reducing administration of the HDAC inhibitor, granzyme B (GzB) release levels increase by at least 25 percent as compared to granzyme B (GzB) release levels during administration of the HDAC inhibitor.

38. The method of claim 35, wherein about 24 hours after terminating or reducing administration, relative cytotoxicity levels increase by at least 50 percent as compared to relative cytotoxicity levels during administration of the HDAC inhibitor.

39. The method of claim 35, wherein the HDAC inhibitor is N-hydroxy-4-(3-methyl-2-phenyl-butyrylamino)-benzamide.

40. The method of claim 35, wherein the method includes administering and terminating administration of the HDAC inhibitor on alternating days.

41. The method of claim 40, wherein administering and terminating administration of the HDAC inhibitor on alternating days allows washout of the HDAC inhibitor to occur between each administration thereof.
